(19) United States
(12) Patent Application Publication
(10) Pub. No.: US 2004/0261373 A1
(43) Pub. Date: Dec. 30, 2004

(54) Method of using medical wrap in continuous form

(75) Inventors: Michael L. Bowen, Mansfield, TX (US); Sergio Diaz de Leon, Clayton, NC (US); Weiping Lin, Mooresville, NC (US); Nick Carter, Mooresville, NC (US)

Correspondence Address: WOOD, PHILLIPS, KATZ, CLARK & MORTIMER, 500 W. MADISON STREET, SUITE 3800, CHICAGO, IL, 60661, US

(73) Assignee: Polymer Group, Inc.

(21) Appl. No.: 10/797,368

(22) Filed: Mar. 10, 2004

Related U.S. Application Data
(60) Provisional application No. 60/453,391, filed on Mar. 10, 2003.

(51) Int. Cl. A61L 2/08; B65B 9/00; B65H 81/00; B32B 1/00

(52) U.S. Cl. 53/477; 422/26; 604/317; 53/425; 53/459

(57) ABSTRACT

The present invention is directed to a method of using a nonwoven medical wrap, and more specifically, to a method of supplying a medical wrap in a continuous form for a more convenient means of wrapping surgical instruments. In a first embodiment, the medical wrap may be in rolled form comprising one or more plies that may be detached from the roll in user determined lengths.

In a second embodiment, the medical wrap may be continuously folded back and forth in a fan-fold or pleated formation so as to form a stack of interconnected continuous sheets. Further, the sheets may be perforated along the folds so that more than one sheet may be detached from the rest of the stack.

### TECHNICAL FIELD

The present invention generally relates to a method of using a medical wrap, and more specifically, to a method of supplying a medical wrap in a continuous form for a more convenient means of preparing materials for sterilization procedures.

### BACKGROUND OF THE INVENTION

Medical wraps are known in art to have been used in both disposable and re-usable discontinuous sheet forms. Typically, medical wraps are used in medical care facilities to envelope surgical equipment during sterilization procedures. Re-usable wovens comprising polyester/cotton blends have been utilized whereby they are laundered after every use and re-used for a finite period of time. U.S. Pat. No. 4,822,667, to Goad et al., hereby incorporated by reference, is representative of one such re-usable woven sterilization wrap.

For the purposes of convenience, and to improve cost efficiency, disposable nonwoven wraps are often used. Nonwoven disposable medical wraps are often made available in sheet forms of a pre-determined size. Traditionally, one or more sheets are utilized during sterilization procedures. Often surgical instruments, including instrument trays are wrapped a first time with a first sheet and then wrapped a second time with a second sheet. U.S. Pat. No. 5,635,134, to Bourne, et al., refers to a single step method of wrapping surgical instruments, which entails the use of a first and second nonwoven sterilization wraps that have been joined to one another.

Disposable medical wraps have been made available in single and double sheet form, routinely packaged in pre-cut sizes, such a small and large. Typically, the single or double sheet forms are dispensed from a box one at a time and prepared for use. A need remains for a more expedient and accommodating method of preparing materials for sterilization procedures, wherein the medical wrap is made available in a continuous form, wherein one or more finite length sheets may be dispensed at one time.

### SUMMARY OF THE INVENTION

The present invention is directed to a method of using a nonwoven medical wrap, and more specifically, to a method of supplying a medical wrap in a continuous form for a more convenient means of wrapping surgical instruments. In a first embodiment, the medical wrap may be in rolled form comprising one or more plies that may be detached from the roll in user determined lengths. Further, the roll may be perforated, or otherwise weakened along predetermined lengths by alternate means, whereby the perforations may be located in close proximity to one another so as to create shorter sheet segments that provide the user with the option of detaching a single short segment or multiple segments.

The medical roll may be used in a freestanding state or utilized with a means for dispensing the wrap. Dispensing means may include a box to house the roll, wherein the box comprises a serrated outer edge that is used to detach a portion of the wrap from the remainder of the housed roll. Additionally, the medical roll may be adjoined to a table, whereby medical personnel may pull a desired portion of the roll across and onto the table and detach the desired potion from the remainder of the roll.

In accordance with the present invention, the medical roll may be utilized in conjunction with mechanical cutting and/or sealing device. For example, when the medical roll comprises more than one ply within the roll, the desired length of the plies may be positioned within a cutting device that not only mechanically detaches the plies from the remainder of the roll, but also thermally seals the edges of the plies to adjoin them at the cutting edge.

In a second embodiment, the medical wrap may be continuously folded back and forth in a fan-fold or pleated formation so as to form a stack of interconnected continuous sheets. Further, the sheets may be perforated along the folds so that more than one sheet may be detached from the rest of the stack.

The use of medical wrap in continuous form is more convenient than dispensing and arranging single sheets one at a time. Also, continuous forms such as rolls are easier to store than multiple boxes of single sheets of predetermined sizes. Further, a continuous roll form may comprise a larger quantity of the medical wrap than individually packaged boxes, needing to be replaced less often. In addition, using medical wrap is in a continuous form decreases the amount of wasted material since the user may determine the amount of wrap to detach from the roll based on the size of the object to be wrapped.

### BRIEF DESCRIPTION OF THE DRAWINGS

FIG. 1 is a diagrammatic view of a single layer perforated medical wrap in a continuous roll form;

FIG. 2 is a diagrammatic view of a double layer perforated medical wrap in a continuous roll form;

FIG. 3 is a diagrammatic view of a medical wrap in a continuous roll form contained within a dispensing means;

FIG. 4 is a diagrammatic view of a single layer perforated medical wrap in a continuous roll adjoined to a table;

FIG. 5 is a diagrammatic view of a double layer medical wrap in a continuous roll form that is drawn through a mechanical cutting and sealing device; and

FIG. 6 is a diagrammatic view of a fan-fold medical wrap in a continuous form.

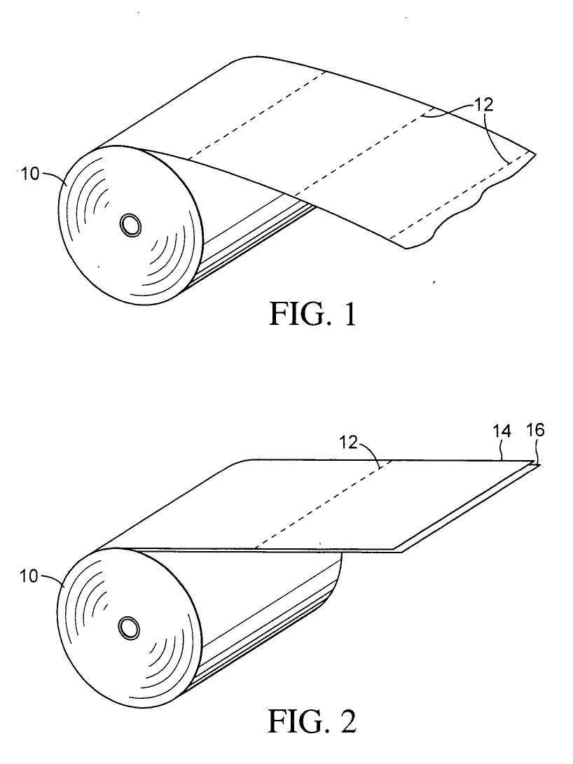

### DETAILED DESCRIPTION

While the present invention is susceptible of embodiment in various forms, there will hereinafter be described, presently preferred embodiments, with the understanding that the present disclosure is to be considered as an exemplification of the invention, and is not intended to limit the invention to the specific embodiments disclosed herein.

The present invention is directed to a method of using a nonwoven medical wrap that is supplied in a continuous form. Manufacture of a nonwoven fabric embodying the principles of the present invention can be comprised of fibers or filaments selected from natural or synthetic composition, of homogeneous or mixed fiber length. Suitable natural fibers include, but are not limited to, cotton, wood pulp and viscose rayon. Synthetic fibers, which may be blended in whole or part, include thermoplastic and thermoset polymers. Thermoplastic polymers suitable for blending include polyolefins, polyamides and polyesters. The thermoplastic polymers may be further selected from homopolymers; copolymers, conjugates and other derivatives including those thermoplastic polymers having incorporated melt additives or surface-active agents. The profile of the fiber or filament is not a limitation to the applicability of the present invention.

In addition, the nonwoven fabric of the present invention may comprise two or more similar or dissimilar layers that are positioned face-to-face and bonded together by suitable means known in the art. Such nonwoven fabrics are represented in U.S. Pat. No. 6,381,817 and No. 6,516,502, to Moody III, hereby incorporated by reference. It is also contemplated that the nonwoven fabric may comprise a film layer, wherein the film layer may be directly extruded onto a nonwoven substrate or positioned face-to-face with a nonwoven substrate and bonded together by suitable means known in the art. Further, the film layer and nonwoven layer may remain un-bonded in a continuous form or partially bonded depending on the desired end-use.

In accordance with the present invention, a method for using a medical wrap in a continuous form is more convenient than boxes that dispense single sheets. Single predetermined size sheets consume more storage due to the various sizes that need to be kept in stock so as to accommodate objects of various sizes in sterilization procedures. Continuous form medical wraps, such as roll forms, require less storage space given that the user may determine the appropriate amount of medical wrap that is necessary for proper coverage of instruments in sterilization procedures. Also, continuous roll forms don't need to be replaced as often as the single sheet boxed form, due to the amount of medical wrap that can be contained in a single roll. In addition, using medical wrap is in a continuous form decreases waste since the user may determine the amount of wrap to detach from the roll based on the size of the object to be wrapped. For example, large objects that are of awkward size and shape or small objects that require minimal wrap material aren't easily wrapped by single sheets that are made available in predetermined sizes. Generally, a certain amount of waste is inevitable in order to accommodate such objects.

The medical wrap may be supplied in a continuous sheet that is entirely planar without any folds. It has also been contemplated that the medical wrap comprise one or more folds and then wound to be supplied in a continuous roll form. In one embodiment, the medical wrap may be C-folded, wherein both edges that run the length of the fabric are folded in toward the center of the fabric. U.S. Pat. No. 5,569,228 and No. 5,366,145, hereby incorporated by reference, are two representative methods of C-folds that can be used in accordance with the invention. Further, one or both of the folded edges of the C-folded wrap may be perforated. In another embodiment, the medical wrap may be folded in half along the length of the fabric and then wound on a roll to be supplied in a continuous form. Again, the medical wrap may optionally be perforated along the fold and then perforated at pre-determined lengths transversely throughout the roll.

A medical wrap roll embodying the principles of the present invention, may be used in a freestanding state. Further, FIG. 2 shows a roll comprising transversely located weakened attachment points positioned throughout said roll, such as perforations (12), at predetermined lengths, wherein the perforations may be located in close proximity to one another so as to create shorter sheet segments that provide the user with the option of detaching a single short segment or multiple segments. It is also contemplated that the medical roll comprise more than one layer, as in FIG. 3, wherein a FIG. 3 shows a first layer (14) and a second layer (16) contained within a single roll (10). In addition, the first layer (14) and second layer (16) are perforated (12) to assist with the detachment of the selected potions from the rest of the roll.

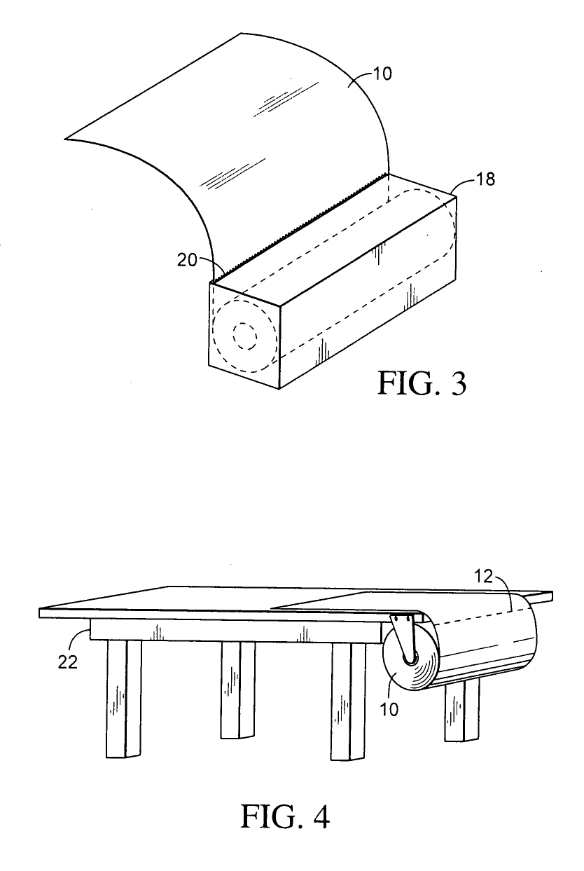

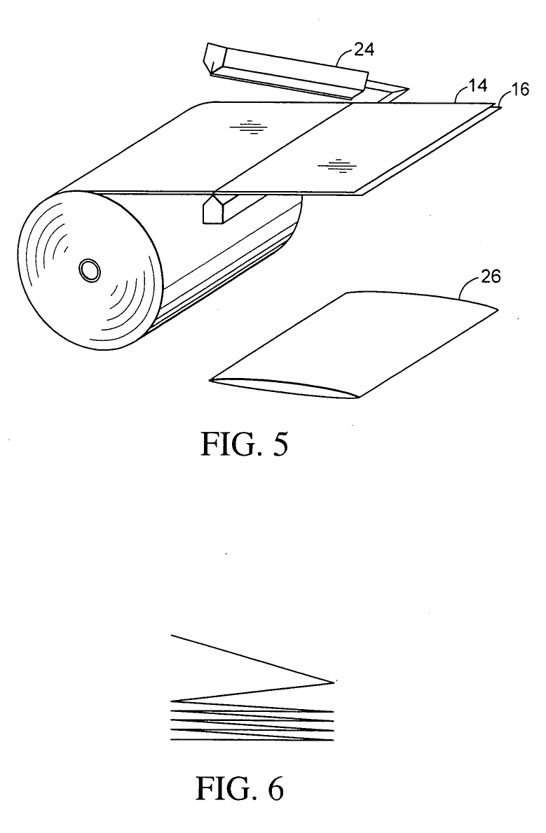

It is also in the purview of the invention that the roll of medical wrap be utilized with a means for dispensing the wrap, illustrated in FIG. 4. FIG. 4 shows a dispensing means that includes a box (18) to house the roll (10), wherein the box comprises a serrated outer edge (20) that is used to detach a portion of the wrap from the remainder of the housed roll (10). Additionally, the medical roll may be adjoined to a table, shown in FIG. 5. This method of use allows medical personnel to pull a desired portion of the roll over the table (22) or work station and detach the desired potion via perforations (12) or other detachment means from the remainder of the roll (10).

FIG. 5 illustrates a method of mechanically cutting and/or thermally sealing medical wrap comprising more than one layer. Drawing the first layer (14) and second layer (16) through the cutting and sealing device (24) thermally bonds the first and second layers, resulting in a medical wrap that is sealed at both edges (26) as the roll is progressively used. FIG. 6 illustrates another method of utilizing a medical wrap in a continuous form, wherein the wrap in fan-folded and perforated at the folds. This method also allows for detaching more than a single at once.

From the foregoing, it will be observed that numerous modifications and variations can be affected without departing from the true spirit and scope of the novel concept of the present invention. It is to be understood that no limitation with respect to the specific embodiments illustrated herein is intended or should be inferred. The disclosure is intended to cover, by the appended claims, all such modifications as fall within the scope of the claims.

What is claimed is:

1. A method of using one or more layers of medical wrap in a continuous form, wherein said wrap is a rolled form comprising transversely located weakened attachment points positioned throughout said roll, whereby said roll may dispenses multiple sheets at once.

2. A method of using a medical wrap in a continuous form as in claim 1, wherein said roll is housed in a dispensing means.

3. A method of using a medical wrap in a continuous form as in claim 2, wherein said dispensing means is a box.

4. A method of using a medical wrap in a continuous form as in claim 3, wherein said box comprises a serrated outer edge.

5. A method of using a medical wrap in a continuous form as in claim 1, wherein said roll is adjoined to a table.

6. A method of using a medical wrap in a continuous form as in claim 1, wherein said roll is used with mechanical cutting means.

7. A method of using a medical wrap in a continuous form as in claim 6, wherein said cutting means thermally seals the cutting edge.

8. A method of using one or more layers of medical wrap in a continuous form, wherein said wrap is in fan-folded form comprising transversely located weakened attachment points positioned throughout said fan-folded form, whereby said fan-folded form may dispense multiple sheets at once.

9. A method of using a medical wrap in a continuous form as in claim 8, wherein said fan-folded wrap comprises transversely located weakened attachment points positioned throughout said wrap.

10. A method of using one or more layers of medical wrap in a continuous form as in claim 1, wherein said wrap is C-folded and wound to be used in a continuous form.
